(19) United States
(12) Patent Application Publication
(10) Pub. No.: US 2011/0070305 A1
(43) Pub. Date: Mar. 24, 2011

(54) SUSTAINED RELEASE PHARMACEUTICAL COMPOSITION CONTAINING MEBICAR

(75) Inventors: Joseph Schwarz, Markham (CA); Michael Weisspapir, Markham (CA)

(73) Assignee: ALPHARX INC., Markham (CA)

(21) Appl. No.: 12/566,419

(22) Filed: Sep. 24, 2009

(51) Int. Cl. A61K 9/28; C07D 487/04; A61K 31/4188; A61P 25/24; A61P 25/22; A61P 25/18

(52) U.S. Cl. 424/474; 548/303.4; 424/464; 514/393

(57) ABSTRACT

The invention relates to a sustained release pharmaceutical composition, containing mebicar, for the treatment of anxiety, depression and other psychiatric and neurological disorders.

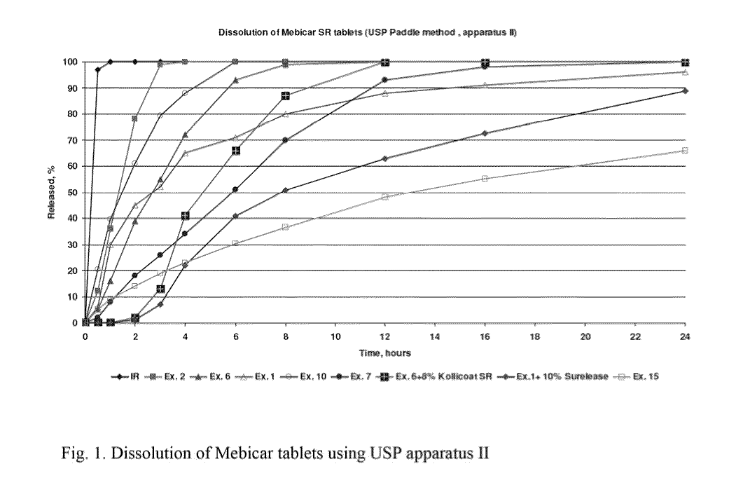

The invention relates to a sustained release pharmaceutical composition, containing mebicar, for the treatment of anxiety, depression and other psychiatric and neurological disorders.

### FIELD OF THE INVENTION

The present invention relates to a sustained release formulation of Mebicar. Particularly, this invention is related to a formulation and dissolution specifications from a sustained release dosage form of Mebicar and improved dosing for a better patient compliance.

### BACKGROUND OF THE INVENTION

Mebicar, as a psychotropic agent, is administered as an injectable solutio or as an immediate release tablet, according to U.S. Pat. No. 4,004,013. Same dosage forms are recommended for the utilization of Mebicar as an anti-anginal drug in U.S. Pat. No. 4,571,403. The usual adult dose is 1-3 tablets of 300 or 500 mg, administered every 4 hours. Mebicar is effective in the treatment of nervousness, psychoses, depression and anxiety and usually is used as a day tranquilizer.

Mebicar has excellent solubility in water (about 50% at ambient temperature) and is soluble in alcohol and most polar organic solvents. The half-life of orally administered Mebicar is short (approximately 3 hours) and is eliminated from the body quickly, thus requiring frequent dosing (every 3-4 hours).

The advantages of extended release formulations are well recognized. The use of sustained release dosage forms permit reduce dose frequencies and improve patient compliance. Nevertheless, existing formulations of Mebicar are immediate release compressed tablets, and the required administration of several tablets 3-4 times daily is inconvenience and can lead to non-compliance.

### SUMMARY OF THE INVENTION

The object of the invention is to provide an oral dosage form containing mebicar, with such dosage form slowly releasing the active component after oral administration. The rate of drug release should be adjusted so to increase dosing intervals from 3-4 hours for immediate release tablets, to 6-24 hours for a slow release formulation.

### DETAILED DESCRIPTION OF THE INVENTION

The invention contemplates a sustained release formulation of Mebicar for oral administration in a tablet form. Slow and sustained drug release from the tablet is regulated by various methods:

a) incorporation of the active component into a polymeric matrix, made of gel forming polymers and developing a gel layer after contact with water media after swallowing. A variety of cellulose derivatives (HPMC, HPC, HEC, CMC), natural gums (xanthan gum, locust been gum, carrageenan) or synthetic polymers (polyethylene oxide (Polyox™), carboxyvinyl polymer (Carbopol™), polyvinyl alcohol, PVA; polyvinylpyrrolidone, PVP and copolymers) can be used; the formed gel slowly dissolves and Mebicar is gradually released.

b) by the inclusion of Mebicar into a water insoluble continuous composition, such as wax, lipid or polymeric matrix. Again, different waxes (natural, such as beeswax, carnauba wax), synthetic (fatty acid esters, e.g., stearyl stearate), lipids such as mono-, di- and triglycerides (glyceryl behenate, glyceryl tristearate), long chain alcohols, such as stearyl alcohol or cholesterol and synthetic polymers (polyvinyl acetate, polymethylmetacrylate and other acrylic acid copolymers) can be compressed into a tablet along with Mebicar and other excipients. The water insoluble material forms a network retaining the drug, and the active component is released gradually after dissolving on contact with water media.

c) Another possible drug release regulatory mechanism is through the formation of a so-called “osmotic pump”. Coating the Mebicar containing tablet with a semi-permeable membrane allows water to enter the interior of the tablet dissolving the drug and other excipients. Due to the water solubility of Mebicar, a high osmotic pressure is developed and drug solution is streamed out of the coating through the pores of the semi-permeable membrane, or through specially drilled holes in the tablet coating.

The sustained release tablet can be additionally coated with a taste masking layer, which can be composed of sugar, water soluble polymers, such as hydroxypropylmethylcellulose (Methocel™ K and E), methylcellulose (Methocel™ A), hydroxypropylcellulose (Klucel®), carrageenan (LustreClear™), acrylic copolymers such as Eudragit® E, vinyl copolymers such as Gantrez® as well as natural gelatin or acacia (Arabic) gum. An enteric coating can also be applied to the matrix tablet.

To prepare the matrix core of the tablet, which is comprised of 500-1500 mg of Mebicar, components can be granulated using water- or solvent based granulation process. Alternatively, components can be granulated by compacting, slugging or extrusion/molding, depending on the properties of matrix forming compound. After drying and milling the dry granulate, it is screened through an appropriate pore sized screen, mixed with other excipients (glidants, lubricants, fillers) and compressed into tablets of an elongated oval or cylindrical shape. It is also possible to prepare a bi-layer tablet core combining immediate release and sustained release components. The formed core may be further coated for taste masking, ease of swallowing, enteric protection or controlled release.

Sustained release means than not more than 90% of the active component is released within the first 2 hours, using a USP paddle dissolution method in apparatus 2 at 50 rpm (USP dissolution test, monograph <711>). Degassed 0.05M Phosphate buffer with pH 6.8 (simulated intestinal fluid without pancreatin, USP) is used as a dissolution medium.

The following non-limiting examples illustrate the invention in greater detail:

Sustained Release Tablet Core Compositions, Based on Gel Forming Polymers.

Examples 1-6 are based on water soluble cellulose ethers, hydroxypropylmethylcellulose (HPMC, Methocel®), hydroxypropylcellulose (HPC, Klucel™) and cellulose ester-carboxymethylcellulose sodium salt (Na CMC, Aqualon).

TABLE 1
Compositions of Mebicar sustained release tablet cores with cellulose
polymers (examples 1-6)

| Component | Example 1 | Example 2 | Example 3 | Example 4 | Example 5 | Example 6 |
|---|---|---|---|---|---|---|
| Mebicar | 900 | 1000 | 1200 | 1000 | 1000 | 1000 |
| Methocel ® K4M CR | 180 | | | | | |
| Methocel ® K15M CR | | 200 | | | | |
| Methocel ® K100M CR | | | 200 | | | |
| Klucel ™ HXF | | | | 100 | 100 | |
| Aqualon CMC Na | | | | | | 250 |
| Povidone ® K-90 | | 10 | 10 | | | |
| Povidone ® K-30 | 10 | | | | | |
| Silicone dioxide | 10 | 5 | 10 | 40 | 40 | 10 |
| Alcohol 95% USP | q.s.* | q.s.* | | q.s.* | —** | |
| Water | | | q.s.* | q.s.* | — | q.s.* |
| Magnesium stearate | 3 | 3 | 5 | 3 | 3 | 3 |
| Core weight (theor.), mg | 1103 | 1218 | 1425 | 1143 | 1143 | 1143 |
| Dissolution time for 80% | 11 hr | 16 hr | 22 hr | 2.5 hr | <0.5 hr | 6 hr |
| release | | | | | | |

*granulating solvent, totally evaporated after drying
**prepared by compaction of dry mixture

All components are mixed and granulated with the selected solvent, using an appropriate granulator. The formed granulation are dried at 60° C. using a forced air oven at 60° C. or fluid bed drier at 50° C. The dry granulation is milled, screened through an 18-mesh screen, mixed with a lubricant (magnesium stearate) and compressed into tablets. Formulations of examples 4 and 5 are identical, but granulation for example 5 tablet cores are prepared by slugging/compaction, followed by milling, screening and tabletting. Examples 6-10 are based on high molecular weight synthetic polymers, polyethylene oxide (Polyox™) and carboxyvinyl polymer-slightly crosslinked polyacrylic acid (Carbopol™).

TABLE 2
Compositions of Mebicar sustained release tablet cores with water
soluble or water swellable synthetic polymers (examples 6-11)

| | Exam- | Exam- | Exam- | Exam- | Exam- |
|---|---|---|---|---|---|
| Component | ple 7 | ple 8 | ple 9 | ple 10 | ple 11 |
| Mebicar | 500 | 900 | 1000 | 900 | 1250 |
| POLYOX WSR N-301 | 100 | | | | |
| POLYOX WSR | | 100 | | | |
| Coagulant | | | | | |
| POLYOX WSR N-303 | | | 200 | | 150 |
| Carbopol ™ 71P | | | | 100 | |
| Methocel ® K100 LV | | | 100 | | |
| Povidone ® K-30 | 10 | | | | 20 |
| Silicone dioxide | 30 | 30 | 30 | 40 | |
| Alcohol 95% USP | q.s.* | q.s.* | | —** | q.s.* |
| Water | | | q.s.* | — | |
| Magnesium stearate | 3 | 3 | 3 | 10 | 5 |
| Core weight (theor.), mg | 643 | 1033 | 1333 | 1050 | 1425 |
| Dissolution time for 80% | 14 hr | 6 hr | 16 hr | 4.5 hr | 9 hr |
| release | | | | | |

*granulating solvent, totally evaporated after drying
**prepared by compaction of dry mixture

All components are mixed and granulated with the selected solvent, using an appropriate granulator. The formed granulation is dried at 50° C. using a forced air oven at 60° C. The dry granulation is milled, screened through an 18-mesh screen, mixed with a lubricant (magnesium stearate) and compressed into tablets. A formulation of tablet cores of example 10 are prepared by slugging compaction, followed by milling, screening, lubricating and tabletting.

TABLE 3
Compositions of Mebicar SR tablet cores with water insoluble matrix
(examples 12-16)

| | Exam- | Exam- | Exam- | Exam- | Exam- |
|---|---|---|---|---|---|
| Component | ple 12 | ple 13 | ple 14 | ple 15 | ple 16 |
| Mebicar | 800 | 800 | 1000 | 800 | 1000 |
| Tristearin (Precirol ™ | 200 | 200 | | | |
| ATO) | | | | | |
| Stearyl alcohol | | | 250 | | |
| Eudragit ® RL | | | | 200 | |
| Kollidon ™ SR | | | | | 250 |
| Methocel ® K100 LV | | | 50 | | |
| Alcohol 95% USP | q.s.* | —** | q.s.* | q.s.* | q.s.* |
| Magnesium stearate | | | | 10 | 10 |
| Core weight (theor.), mg | 1000 | 1000 | 1300 | 1010 | 1260 |
| Dissolution time for | 5 hr | <1 hr | 6 hr | >24 hr | 16 hr |
| 80% release | | | | | |

*granulating solvent, totally evaporated after drying
**prepared by compaction of dry mixture

All components are mixed and granulated with the selected solvent, using an appropriate granulator. The formed granulation is dried at 50° C. using a forced air oven at 60° C. The dry granulation is milled, screened through an 18-mesh screen, mixed with a lubricant (magnesium stearate) and compressed into tablets. A formulation of tablet cores of example 13 are prepared by compaction, followed by milling, screening and tabletting.

All prepared tablet cores have high hardness (10-30 kP), low friability and can be coated using standard coating techniques. The coating can be designed to be soluble for taste masking, enterosoluble for stomach protection or insoluble but permeable for dissolution rate regulation.

TABLE 4
Coating of Mebicar SR tablet cores

| Coating compound | Coating objective | Level, % | Solvent |
|---|---|---|---|
| Hypromellose (Opadry ® II, Colorcon) | taste masking | 2-3 | water |
| Carrageenan (LustreClear ™, FMC) | taste masking | 2-4 | water |
| Dimethylaminnoethylmetacrylate (Eudragit E, Degussa) | taste masking | 1.5-3 | organic |
| Metacrylic acid copolymer (Eudragit ® L30D, Degussa) | enteric coating | 4-9 | water |
| Hypromellose phthalate (HPMCP 55, Shin-Etsu) | enteric coating | 5-10 | organic |
| Ethylcellulose (Surelease ™ NG, Colorcon) | delayed release | 4-12 | water |
| Polyvinyl acetate (Kollidon SR 30D, BASF) | osmotic pump | 5-15 | water |
| Cellulose acetate (CA-398-10NF, Eastman) | osmotic pump | 7-12 | organic |

Sustained release tablets of Mebicar can be administrated orally 1-2 times a day or as recommended by a specialist, with a maximal single dose of 500-3000 mg. and a maximal daily dose of 10 g.

1. A pharmaceutical oral dosage form composition, comprising 500 to 1500 mg of mebicar (2,4,6,8-tetramethyl-2,4,6,8-tetraazabicyclo-[3.3.0]-octanedione-3,7) wherein said dosage form provides sustained release of mebicar for 2-24 hours after oral administration.

2. A pharmaceutical oral dosage form as set forth in claim 1, wherein said dosage form is a tablet.

3. A pharmaceutical oral dosage form as set forth in claim 2, wherein said tablet is a matrix tablet.

4. A pharmaceutical oral dosage form as set forth in claim 2, wherein said tablet is a coated tablet.

5. A pharmaceutical oral dosage form as set forth in claim 3, wherein said matrix is formed by a gel-forming component.

6. A pharmaceutical oral dosage form as set forth in claim 3 wherein said matrix is formed by a water insoluble component.

7. A pharmaceutical oral dosage form as set forth in claim 4 wherein said tablet is coated with a water soluble coating.

8. A pharmaceutical oral dosage form as set forth in claim 4 wherein said tablet is coated with a water insoluble coating.

9. A pharmaceutical oral dosage form as set forth in claim 5 wherein said gel-forming component is selected from a group consisting of hydroxypropylmethylcellulose, hydroxypropylcellulose, hydroxyethylcellulose, hydroxypropyl ethylcellulose, methylcellulose, xantham gum, alginate, carrageenan, locust bean gum, guar gum, polyethylene oxide, carboxymethylcellulose or cross-linked carboxyvinyl polymers.

10. A pharmaceutical oral dosage form as set forth in claim 6 wherein said water insoluble component is selected from a group consisting of glyceryl behenate, cetostearyl alcohols, waxes, mono-, di- and triglyceryl stearates, polyvinylacetate, polyvinylcaprolactone, polyacrylates or polymethacrylates.

11. A pharmaceutical oral dosage form as set forth in claim 1, providing 15-95% release within the first 4 hours of dissolution in water media, and not less than 80% release within 24 hours of dissolution when tested using USP dissolution apparatus II.

12. A pharmaceutical oral dosage form as set forth in claim 1, administrated to patients requiring treatment for anxiety, psychoses, stress or depression.
